(19) United States
(12) Patent Application Publication
(10) Pub. No.: US 2011/0257542 A1
(43) Pub. Date: Oct. 20, 2011

(54) System Method and Device for Performing Heat Stress Tests

(76) Inventors: Brian Russell, Crownsville, MD (US); Stephen Kent, Auckland (NZ); Christopher M. Solomon, Auckland (NZ)

(21) Appl. No.: 13/087,540

(22) Filed: Apr. 15, 2011

Related U.S. Application Data
(60) Provisional application No. 61/324,414, filed on Apr. 15, 2010.

(51) Int. Cl. A61B 5/02

(52) U.S. Cl. 600/500

(57) ABSTRACT

A system, method and device for testing for heat stress of a person is provided. In one embodiment, the method includes determining a first heart rate of the person at the end of a first time period during which a heart rate of the person maintained at least a first predetermined heart rate; after the first time period, determining a second heart rate of the person at the end of a second time period during which the heart rate of the person did not exceed a second predetermined heart rate; wherein the first predetermined heart rate is greater than the second predetermined heart rate; determining that the heart rate of the person transitioned from at least the first predetermined heart rate to no greater than the second predetermined heart rate within a predetermined transition time period; determining a heart rate recovery by subtracting the second heart rate from the first heart rate; determining a heart rate recovery ratio by dividing the heart rate recovery by a heart rate recover baseline; and providing a notification if the heart rate recovery ratio is beyond a threshold.

### CROSS REFERENCE TO RELATED APPLICATIONS

This application claims priority to U.S. Provisional Application No. 61/324,414, filed Apr. 15, 2010, which is hereby incorporated by reference in its entirety for all purposes.

### FIELD OF THE INVENTION

The present invention generally relates to physiological data processing and more particularly, to a system, method and device for performing heat stress tests of a person.

### BACKGROUND OF THE INVENTION

Monitoring vital signs is traditionally done on supine patients at rest. Field based measurements are typically done with a care giver or researcher controlling the person's position and degree of movement in order to minimize movement artifacts such as orthstatic changes and effects on the body due to work effort and orientation. Normally tests are performed under various conditions in a clinic manually, using such devices as blood pressure cuffs or using treadmills and stop watches for exertion fitness tests.

Measuring vital signs over time (in the field) provides more useful information to allow an understanding of a person's physiological state. However, body position and activity level are key factors that affect a person's vital signs and hence the interpretation thereof.

Information of the biomechanical context of a person allows the person's vital signs to be measured and interpreted remotely. Biomechanical sensors include, for example, tri axial accelerometers and gyroscopes which determine the posture and activity level of a person. Biomechanical sensors which are enclosed in or time synchronised to a vital sign monitor afford the opportunity to take measurements that, until now, would not be practical or useful because the person's movement or posture could have a greater effect than the variations sought. In contrast, some embodiments of the present invention can determine a normal state of the person under different activity levels and postures and hence determine an abnormal state.

These and other advantages may be provided by one or more embodiments of the present invention.

### BRIEF DESCRIPTION OF THE DRAWINGS

The invention is further described in the detailed description that follows, by reference to the noted drawings by way of non-limiting illustrative embodiments of the invention, in which like reference numerals represent similar parts throughout the drawings. As should be understood, however, the invention is not limited to the precise arrangements and instrumentalities shown. In the drawings:

FIG. 1 is a flow chart of a process, in accordance with an example embodiment of the present invention.

FIG. 2 is a graphic representation of heart rate and activity levels, in accordance with an example embodiment of the present invention.

FIGS. 3a-b depict a biometric system, that may be used to collect (and process data), in accordance with an example embodiment of the present invention.

### DETAILED DESCRIPTION OF ILLUSTRATIVE EMBODIMENTS

In the following description, for purposes of explanation and not limitation, specific details are set forth, such as particular networks, sensor, algorithm, communication systems, computers, terminals, devices, components, techniques, data and network protocols, software products and systems, operating systems, development interfaces, hardware, etc. in order to provide a thorough understanding of the present invention.

However, it will be apparent to one skilled in the art that the present invention may be practiced in other embodiments that depart from these specific details. Detailed descriptions of well-known networks, communication systems, sensor, algorithm, computers, terminals, devices, components, techniques, data and network protocols, software products and systems, operating systems, development interfaces, and hardware are omitted so as not to obscure the description.

A person's physiology changes based on speed of movement, level of activity and posture. Embodiments of the present invention address the issue of automatically testing various physiological states when using sensors for short term and long term (in the field) monitoring of bioelectric signals of a person. When a person is remote, the clinician or coach cannot make a manual assessment of the person's posture or the time at which a certain event occurred. Embodiments of the present invention provide a novel way to remotely determine these values by using a combination of biomechanical sensors, physiological sensors and algorithms that process these values over time.

Heat stress and heat strain are dangerous events. Being able to measure these on a person can save lives. Non invasive means of measuring these events are preferred as they are likely to be used more. Heat stress is usually determined via core temperature. The problem with this process is that measurements using ingested pills, intravenously or rectal are not practical for field applications involving the general population. A person's core temperature rises due to infection, exercise, body mass, height, solar loading, humidity, wind, clothing insulation, and/or other factors. Heat related illnesses are also affected by sleep hygiene, previous heat illness, physical short term fatigue, acclimatisation, genetic predisposition and hydration.

Embodiments of the present invention process data of a combination of activity sensors, skin temperature sensors and heart rate sensors with an algorithm to determine the changes in heart rate (HR) for various activity levels to determine cardiac drift and additionally to determine the reduction in heart rate recovery post exercise. These factors may then be processed to determine the risk of heat stress to a person.

Blood is circulated in the body for thermal regulation and energy systems. Typically an adult will circulate six L/min of blood for energy systems including transportation of gases (e.g. CO2, O2) and fuels (e.g. Glycogen). The core body temperature of a person is maintained initially through increased blood flow up to 14 L/min and vasodilatation of blood vessels to the skin. If the core temperature continues to increase then sweating will occur. The increased blood flow will lead to cardiac drift (an increase in heart rate) and will also reduce heart rate recovery (HRR) after exercise. The reason for reduced HRR is that the heart continues to pump blood (the additional 8 L/min) to peripheral skin to maximise heat loss.

Algorithms of embodiments of the present invention can be used to process data while a person is carrying out random events (or exercises) or is performing requested (known) behaviour. For example, a person at risk may be asked to stop for one minute so that certain trigger criteria (discussed below) are met.

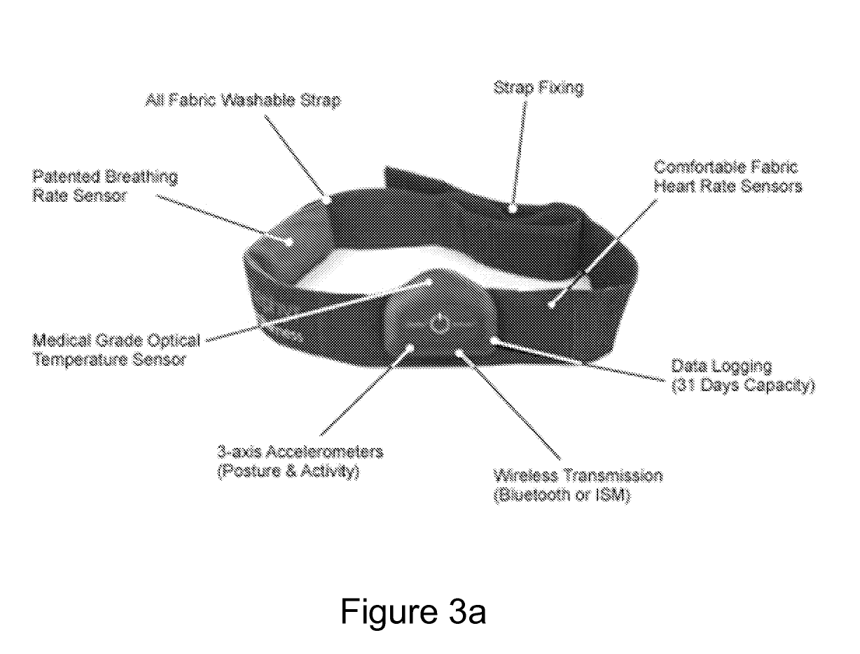

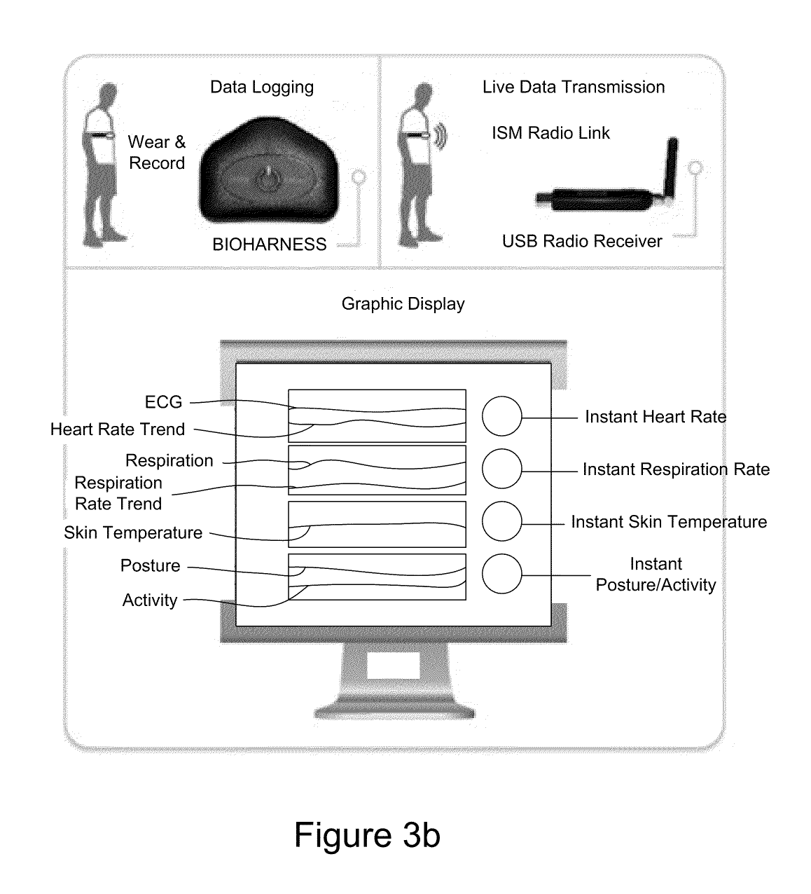

The data used by embodiments of the present invention may be collected and processed by a device called the BioHarness, which is commercially available and manufactured by Zephyr Technology of Annapolis, Md. See FIGS. 3a-b. The device measures heart rate, breathing rate, temperature, activity and posture, is battery powered and worn around the chest (e.g., via a strap). The BioHarness includes a Bluetooth wireless transceiver, processor, and internal memory. The person may wear the device at home and/or work (or in a clinic environment). The data from the biomechanical and physiological sensors (and in some embodiments, environmental sensors) is regularly collected and stored in memory. Upon detection of certain physiological data, the algorithm processes the data to determine the risk of heat stress for the person.

Example heat stress algorithm

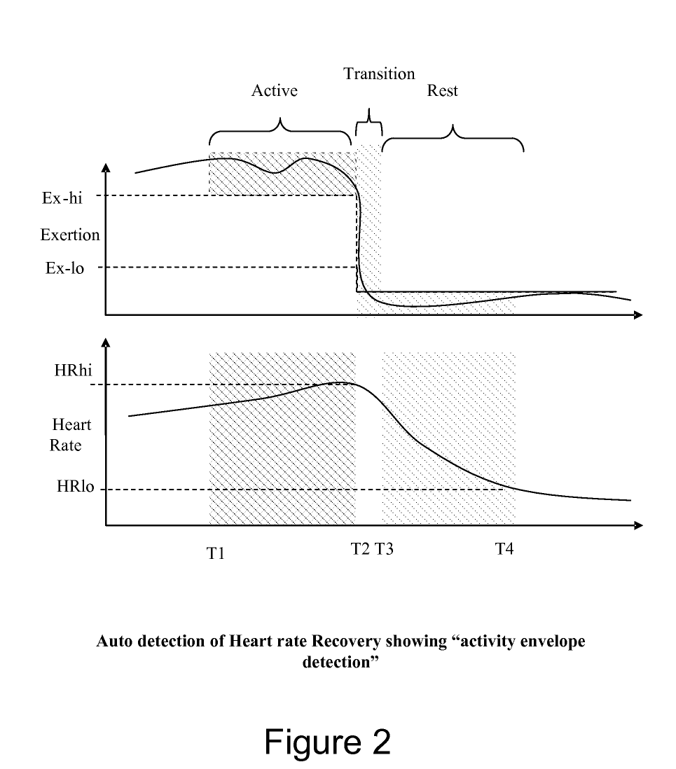

The algorithm below illustrates how the biomechanical sensors measuring activity are used to determine and trigger an automatic measurement on heart rate recovery. An algorithm of one example embodiment may use the following variables referenced in FIG. 2.

Heart Rate Recovery or HRR, is the decrease in heart rate from the time activity stops to a predetermined time. Typically, measurements are thirty seconds, one minute, five minutes, and ten minutes after activity stops. In some embodiments of the present invention, the algorithm may be executed at such times (e.g., when T4 (see FIG. 2) is thirty seconds, one minute, two minutes, five minutes, and ten minutes (after activity stops)).

Transition time is the time period during which the heart rate (HR) transitions from a high (e.g., HRhi in FIG. 2) to a resting HR (e.g., Hrlo in FIG. 2). When the Exertion falls below a high threshold , a clock (or timer) is started. If the measured Exertion rate fails to reach the lower threshold (Hrlo) within an allotted period of time, the entire envelope (i.e., test cycle) must be discarded and new activity envelope detection cycle will begin anew.

HRRbaseline is a previous HRR under controlled conditions where core temperature was acceptable, or based on crowd sourced data for that persons group of age, weight, fitness level, level of exercise per week, etc.

Exertion is the level of exertion the person is under taking. Various measures of exertion can be used such as activity in vector magnitude units, heart rate, breathing rate, activity level (how fast running, swimming, or jumping, etc.), body temperature, speed, power, altitude and/or a distance covered (e.g., walked, run). Thus, the algorithm can use one or more of these to trigger processing of data to determine when the person is above an exertion trigger level or below a certain level defined as “resting”.

Ex-hi is the exertion level which a person must exceed for given period in order for the test to be triggered (i.e., for processing of subsequent data). Ex-hi is thus a level of exertion required to instigate a high level of physical loading.

Ex-lo is the exertion level at which a person must stay below in order to meet the definition of “resting” and to provide data sufficient for heat stress test under some example embodiments. The resting activity level is required for a set time after activity stops in order to maximise the heart rate recovery process and give a constant physiological loading for recovery between tests.

Consecutive tests can be performed for various reasons. Short term consecutive tests over, for example, one day may be used as measure of core temperature increase. Consecutive tests over a year may be used to determine the level of fatigue where an increasing HRR number is an indication of increasing fitness.

HRR_threshold_ratio is the ratio of HRRbaseline to a newly computed HRR. By knowing a baseline HRR, the heat stress risk for a newly computed HRR can be more accurately assessed (than if no baseline HRR were used).

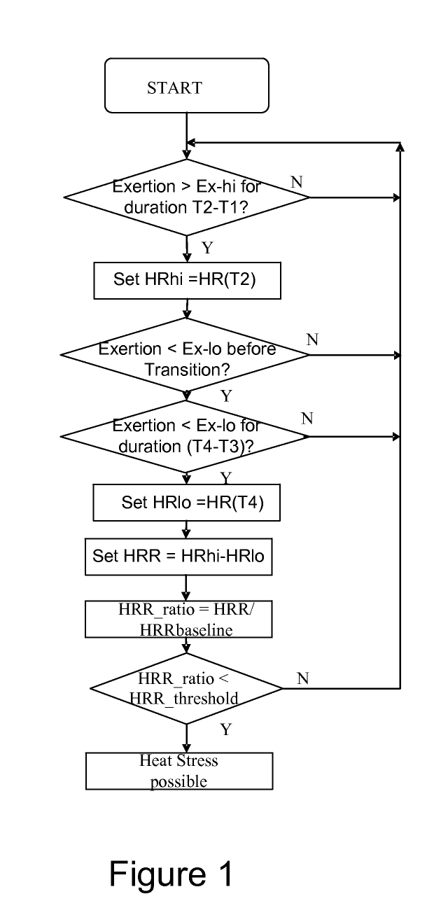

Referring to FIGS. 1 and 2, an algorithm according to an example embodiment of the present invention may perform the following processes:

1. If Exertion is greater than Ex-hi for a duration (T2−T1) then set HRhi=HR (T2) (i.e., the heart rate at time T2)

2. Then if Exertion is below Ex-lo before Transition time, continue with the following steps and if not return to step one.

3. Then if Exertion is below Ex-lo for time (T4−T3) then set Hrlo=HR (T4) (i.e., the heart rate at time T4)

4. Then set HRR=HRhi−Hrlo

5. Then set HRR_ratio=HRR/HRRbaseline

6. IF HRR_ratio<HRR_threshold ratio then heat stress is possible.

Probability of heat stress=K*(1−HRR/HRRbaseline); where K is defined for a person based on age, weight, gender, previous heat incidents and/or other factors.

Modifier factor K—Environmental parameters such as humidity, temperature and solar loading in addition to a persons fatigue history, sleep history, calorie input and exercise loading can be entered into the computer system to modify the factor K which subsequently determines the risk of thermal stress.

If heat stress is possible (or probably), a notification (an alert) may be transmitted (e.g., wirelessly) to medical personnel and/or an audible alarm may be sounded to alert the wearer. Thus, the processing and notification may be in real time such as within thirty seconds, one minute, five minutes or ten minutes of the end of the second time period (T4). Alternately, the data may be processed hours, days or weeks later (not in real time).

The present invention may be embodied, at least in part, as a computer system (one or more co-located or distributed computers) or cluster executing one or more computer programs stored on a tangible medium. The algorithm may be executed (and computer system located) local or remote from the user. The algorithm may be executed on a computer system that also includes other functions such a telephone, tablet computer, portable computer, or other device (e.g., an IPhone®, IPad®, or Blackberry®), which may have processing and communications capabilities. The processing may be performed on the computer system, by the processor forming part of the physiological collection (and processing (system) such as the BioHarness (or a device integrated into (or attached to) a garment (e.g., a shirt), or some combination thereof. In one embodiment, a plurality of devices may be worn by a team, participants in a sporting even, or a group of people. Each device may (1) perform the processing and wirelessly send a notification to a computer (designated to receive such notifications from the devices of the group); or (2) transmit heart rate (or other exertion level data) to a computer (designated to receive such data from devices of the group), which performs the above processing.

Thus, one embodiment of the present invention may comprise a method of testing for heat stress of a person that comprises determining a first heart rate of the person at the end of a first time period during which a heart rate of the person maintained at least a first predetermined heart rate; after the first time period, determining a second heart rate of the person at the end of a second time period during which the heart rate of the person did not exceed a second predetermined heart rate; wherein the first predetermined heart rate is greater than the second predetermined heart rate; determining that the heart rate of the person transitioned from at least the first predetermined heart rate to no greater than the second predetermined heart rate within a predetermined transition time period; determining a heart rate recovery by subtracting the second heart rate from the first heart rate; determining a heart rate recovery ratio by dividing the heart rate recovery by a heart rate recover baseline; and providing a notification if the heart rate recovery ratio is beyond a threshold. Determining a heart rate recovery may be performed in real time such as within five minutes of the end of the second time period. Determining a heart rate recovery may be performed by a device carried on or by the person or remotely (by a device that receives the heart rate data via wirelessly). The heart rate recover baseline may be based on the person's age and gender or data previously collected from the person. The method may further comprise determining a probably of heat stress based on the heart rate recovery ratio.

In another embodiment, the device for testing for heat stress of a person may comprise a plurality of sensors communicatively coupled to the person to capture information of a heart rate of the person; a controller in communication with the plurality of sensors to receive data of the heart rate of the person; wherein said controller is programmed to: determine a first heart rate of the person at the end of a first time period during which the heart rate of the person maintained at least a first predetermined heart rate; after the first time period, determine a second heart rate of the person at the end of a second time period during which the heart rate of the person did not exceed a second predetermined heart rate; wherein the first predetermined heart rate is greater than the second predetermined heart rate;determine that the heart rate of the person transitioned from at least the first predetermined heart rate to no greater than the second predetermined heart rate within a predetermined transition time period; determine a heart rate recovery by subtracting the second heart rate from the first heart rate; determine a heart rate recovery ratio by dividing the heart rate recovery by a heart rate recover baseline; and provide a notification if the heart rate recovery ratio is beyond a threshold. The device may further comprise a processor connected to said plurality of sensors and a wireless transceiver; wherein said processor is configured to cause said wireless transceiver to transmit the data of the heart rate of the person to said controller. The processor may form part of a device that is worn or carried by the person and the controller may form part of a device remote from the person: Alternately, for example, the controller may form part of a device that is worn or carried by the person.

In yet another embodiment, the method of testing for heat stress of a person, may comprise determining a first heart rate of the person at the end of a first time period during which the person maintained at least a predetermined first exertion level; determining a second heart rate of the person at the end of a second time period during which the person did not exceed a second exertion level; wherein the first exertion level is greater than the second exertion level; determining a heart rate recovery as the first heart rate minus the second heart rate; determining a heart rate recovery ratio by dividing the heart rate recovery by a heart rate recover baseline; and providing a notification if the heart rate recovery ratio is beyond a threshold. The method may further comprise determining that an exertion of the person transitions from the first exertion level to the second exertion level within a predetermined transition time period and wherein the first exertion level comprises a first heart rate and the second exertion level comprises a second heart rate. Determining a heart rate recovery may be performed in real time and/or be performed by a device carried on or by the person.

It is to be understood that the foregoing illustrative embodiments have been provided merely for the purpose of explanation and are in no way to be construed as limiting of the invention. Words used herein are words of description and illustration, rather than words of limitation. In addition, the advantages and objectives described herein may not be realized by each and every embodiment practicing the present invention. Further, although the invention has been described herein with reference to particular structure, materials and/or embodiments, the invention is not intended to be limited to the particulars disclosed herein. Rather, the invention extends to all functionally equivalent structures, methods and uses, such as are within the scope of the appended claims. Those skilled in the art, having the benefit of the teachings of this specification, may affect numerous modifications thereto and changes may be made without departing from the scope and spirit of the invention.

What is claimed is:

1. A method of testing for heat stress of a person, comprising:
determining a first heart rate of the person at the end of a first time period during which a heart rate of the person maintained at least a first predetermined heart rate;
after the first time period, determining a second heart rate of the person at the end of a second time period during which the heart rate of the person did not exceed a second predetermined heart rate;
wherein the first predetermined heart rate is greater than the second predetermined heart rate;
determining that the heart rate of the person transitioned from at least the first predetermined heart rate to no greater than the second predetermined heart rate within a predetermined transition time period;
determining a heart rate recovery by subtracting the second heart rate from the first heart rate;
determining a heart rate recovery ratio by dividing the heart rate recovery by a heart rate recover baseline; and
providing a notification if the heart rate recovery ratio is beyond a threshold.

2. The method according to claim 1, wherein said determining a heart rate recovery is performed within five minutes of the end of the second time period.

3. The method according to claim 1, wherein said determining a heart rate recovery is performed by a device carried on or by the person.

4. The method according to claim 1, further comprising:
collecting data of the heart rate of the person;
wirelessly transmitting data of the heart rate of the person; and
wherein said determining a heart rate recovery is performed by a computer remote from the person.

5. The method according to claim 1, wherein the heart rate recover baseline is based on the person's age and gender.

6. The method according to claim 1, wherein the heart rate recover baseline is based on data previously collected from the person.

7. The method according to claim 1, further comprising determining a probably of heat stress based on the heart rate recovery ratio.

8. A device for testing for heat stress of a person, comprising:
a plurality of sensors communicatively coupled to the person to capture information of a heart rate of the person;
a controller in communication with the plurality of sensors to receive data of the heart rate of the person;
wherein said controller is programmed to:
determine a first heart rate of the person at the end of a first time period during which the heart rate of the person maintained at least a first predetermined heart rate;
after the first time period, determine a second heart rate of the person at the end of a second time period during which the heart rate of the person did not exceed a second predetermined heart rate;
wherein the first predetermined heart rate is greater than the second predetermined heart rate;
determine that the heart rate of the person transitioned from at least the first predetermined heart rate to no greater than the second predetermined heart rate within a predetermined transition time period;
determine a heart rate recovery by subtracting the second heart rate from the first heart rate;
determine a heart rate recovery ratio by dividing the heart rate recovery by a heart rate recover baseline; and
provide a notification if the heart rate recovery ratio is beyond a threshold.

9. The device of claim 8, further comprising:
a processor connected to said plurality of sensors and a wireless transceiver;
wherein said processor is configured to cause said wireless transceiver to transmit the data of the heart rate of the person to said controller.

10. The device of claim 9, wherein said processor forms part of a device that is worn or carried by the person and said controller forms part of a device remote from the person:

11. The device of claim 8, wherein said controller forms part of a device that is worn or carried by the person.

12. A method of testing for heat stress of a person, comprising:
determining a first heart rate of the person at the end of a first time period during which the person maintained at least a predetermined first exertion level;
determining a second heart rate of the person at the end of a second time period during which the person did not exceed a second exertion level;
wherein the first exertion level is greater than the second exertion level;
determining a heart rate recovery as the first heart rate minus the second heart rate;
determining a heart rate recovery ratio by dividing the heart rate recovery by a heart rate recover baseline; and
providing a notification if the heart rate recovery ratio is beyond a threshold.

13. The method according to claim 12, further comprising determining that an exertion of the person transitions from the first exertion level to the second exertion level within a predetermined transition time period.

14. The method according to claim 12, wherein the first exertion level comprises a first heart rate and the second exertion level comprises a second heart rate.

15. The method according to claim 12, wherein said determining a heart rate recovery is performed in real time.

16. The method according to claim 12, wherein said determining a heart rate recovery is performed by a device carried on or by the person.

17. The method according to claim 12, further comprising:
collecting data of an exertion of the person;
wirelessly transmitting data of the exertion of the person; and
wherein said determining a heart rate recovery is performed by a device remote from the person that receives the data of the exertion of the person via a communication path that includes said wirelessly transmitting.

18. The method according to claim 12, wherein the heart rate recover baseline is based on the person's age and gender.

19. The method according to claim 12, wherein the heart rate recover baseline is based on data previously collected from the person.

20. The method according to claim 12, further comprising determining a probably of heat stress based on the heart rate recovery ratio.
